(19) United States
(12) Patent Application Publication
(10) Pub. No.: US 2007/0055193 A1
(43) Pub. Date: Mar. 8, 2007

(54) Traction system for operating table comprising an automatic control device

(75) Inventor: Philippe Daury, Boulogne (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: FHSURGICAL, an organization of France, Paris (FR)

(21) Appl. No.: 10/987,248

(22) Filed: Nov. 12, 2004

(30) Foreign Application Priority Data
Nov. 14, 2003 (FR) FR 03/13376

(51) Int. Cl. A61F 5/00

(52) U.S. Cl. 602/33

(57) ABSTRACT

The present invention pertains to a traction system (1) for operating table suitable for the orthopedic surgery of a patient's upper and/or lower limb, of the type comprising a mobile support (3) intended to receive said limb to be operated on and extension means (4) enabling application at the end of said limb of a traction force on the proximal part of said limb, characterized in that said system comprises means (5) for the measurement of the traction force applied to said limb, which is linked to an automatic control system (6) modifying the traction force exerted on the limb until reaching an index value relative to the desired extension.

The present invention pertains to traction systems intended for surgical operating tables.

The present invention pertains more specifically to a traction system for operating table suitable for the orthopedic surgery of a patient's upper and/or lower limb, of the type comprising a mobile support intended to receive said limb to be operated on, and extension means enabling the application of a traction force on the end of said limb.

Orthopedic surgery is conventionally performed on operating tables comprising tractions systems used on a patient's lower limbs composed of traction arms comprising mechanical screw tractors. These traction arms are attached to a frame that comprises the elements necessary for supporting the patient's pelvis. These traction arms can be articulated horizontally and vertically. During the entire surgical intervention, the limb to be operated on is subjected to a traction force which must remain constant in order to prevent lesions.

The orthopedic surgical intervention requires that the surgeon has easy and adequate access to the surgical field in order to be able to operate under satisfactory conditions. In order to achieve this, it becomes necessary for the surgeon to displace the limb upon which he is operating during the intervention. The displacement of the limb of course leads to a modification of the extension of the limb in question and thus the traction force exerted on this limb. Although relatively slight, the variation of the extension involved is sufficient to cause lesions.

There was proposed in international patent application WO 94/05227 a traction and wrist-support device to be used during arthroscopic or orthopedic surgery. Said device comprises a support for holding the upper arm, a coupling element for holding the fingers as well as an extendable element connected in a pivoting manner between the support and the coupling element, extension means for increasing or decreasing the length of the extendable element so as to apply to the wrist an essentially constant traction force by the intermediary of regulated extension force applied to said extendable element. According to one configuration of the invention, the traction force is controlled by the surgeon who modifies said force by adjusting, by means of a pressure regulator, the pneumatic or hydraulic pressure to provide the extendable element with the required force.

Nevertheless, the device of patent application WO 94/05227 remains relatively imprecise with regard to the traction force applied. Moreover, such a traction device requires a continuous monitoring of the traction force exerted on the wrist, as well as a rather demanding handling given that the surgeon himself or the medical personnel are responsible for regulating the extension force.

The intention of the present invention is to resolve the drawbacks of the prior art by proposing a traction system in the context of orthopedic surgical operation, which system automatically compensates for the variation in the extension applied to the limb to be operated on subsequent to any modification of the orientation and/or displacement of the limb in question.

The present invention also has as object the proposal of a traction system making it possible to exert on the limb to be operated on the desired extension force in a precise and reliable manner.

The present invention also has as object the proposal to the surgeon of a traction system that can easily be implemented.

In order to attain this object, the present invention is of the type described above and it is remarkable in its broadest sense in that the traction system comprises means for measuring the traction force applied to said limb to which means is connected an automatic control device modifying the traction force applied to said limb until reaching an index value relative to the desired extension.

Said support is preferably mobile in rotation around an axis coinciding essentially with a pivoting axis of said limb.

The means for the measurement of the applied force advantageously consist of a dynamometer.

Said dynamometer is advantageously constituted by piezoelectric sensors.

Said automatic control device advantageously comprises means for the comparison of the value of the force applied to said limb with the reference value of said force stored in a memory unit of said automatic control device.

Said automatic control device advantageously comprises means for controlling the displacement of the extension means in a manner such that said extension means exert on said limb the index extension value.

Said system advantageously moreover comprises visual and/or audio alarm means triggered upon detection by said automatic control device of a variation of the force applied to the limb in relation to the given index value.

The present invention also pertains to an operating table comprising at least one traction system suitable for orthopedic surgery as defined above.

According to one particular mode of implementation of the invention, said operating table has an elongation articulated around a pivot perpendicular to said table corresponding essentially to the position of the patient's hip, said elongation comprising extension means enabling application at the end of a patient's leg of a traction force and means for measuring said traction force applied to said leg which is linked to an automatic control device modifying said traction force until reaching an index value relative to the desired extension.

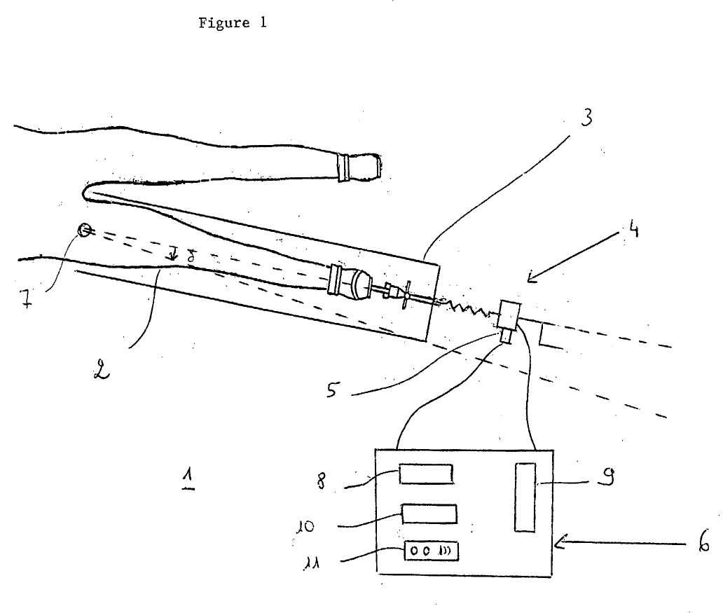

Better understanding of the invention will be obtained from the description provided below for purely explanatory purposes of one mode of implementation of the invention with reference to FIG. 1 which illustrates a schematic view of a traction system according to the invention.

FIG. 1 illustrates a traction system (1) intended for an operating table to be used for orthopedic surgical operations.

In this example of implementation, the traction system (1) according to the invention is used in the context of an operation on a patient's leg (2).

Said traction system (1) comprises a mobile support (3) on which rests the leg (2) to be operated on. Extension means (4) are attached to the end of the leg (2) in order to apply a traction force on the proximal part of the leg (2).

The support (3) is advantageously mobile in rotation around an axis (7) coinciding essentially with a pivoting axis of the leg (2).

The traction system (1) moreover comprises means (5) for measuring the traction force applied to the leg (2). The measurement means (5) advantageously consist of two piezoelectric sensors constituting a dynamometer which makes it possible to set an index value when the traction force applied to the end of the leg (2) corresponds to the desired extension.

The index value is then recorded in a memory unit (9) of an automatic control device (6). Said automatic control device (6) furthermore comprises, in addition to the memory unit (9), means (8) for comparison of the value of the force applied to said limb with the index value of the force stored in the memory unit (9). It also comprises means (10) intended for controlling the displacement of the extension means.

Thus, connected to the dynamometer (5), said automatic control device (6), in the case of variation of the traction force applied to the leg (2) in relation to the index value recorded in said memory unit (9), modifies the applied traction force in order to return to the set extension index value.

More precisely, after having recorded in advance a traction index value in the memory unit (9) of said automatic control device (6) by means of said dynamometer (5), the operation of correction of the traction force is implemented as follows. The traction force is measured on a continuous basis by means of said dynamometer (5). The value obtained for the applied force is transmitted to said automatic control device which will compare the value of said force with the recorded index value. Said operation is performed by said comparison means (8) constituting said automatic control device (6). If the comparison step leads to the observation of a deviation between the value of the applied force and the index value stored in memory subsequent to an angular displacement δ of the leg (2) being operated on, a command is transmitted to the control unit (10) which will modify the traction force exerted on the end of the leg (2) until returning to the recorded index value.

It is understood that the surgeon could decide to not implement an automatic correction of the applied traction force. He could decide to intervene on the extension means (4) in a manner so as to return to the index value stored in memory by inactivating the control means (10) of the displacement of said extension means (4).

According to one advantageous mode of implementation, the automatic control device (6) comprises visual and/or audio alarm devices (11). Thus, upon detection by said automatic control device (6) of a variation in the force applied to the leg in relation to the index value recorded in the memory unit (9), the visual and/or audio alarms are triggered so as to warn the surgeon in the case of exceeding or diminishment of the traction force applied in relation to the recorded index value.

The audio alarm devices are advantageously electroluminescent diodes. According to one variant of implementation of the invention, the automatic control device (6) can comprise a supplementary light indicator of the LED type in order to keep the surgeon informed at all times of the correspondence of the value of the applied traction force with the index value. In particular, in the absence of variation of the traction force applied to the leg (2), the light indicator, e.g., green in color, would be active. In the case of variation of the traction force, the light indicator would go out and the alarm device (11) would take over. Upon return to the index value of the traction force, the light indicator would again be illuminated.

The invention was described above as an example. It is understood that the expert in the field would be capable of implementing different variants of the invention without thereby going beyond the framework of the patent.

1-9. (canceled)

10. A traction system applicable to a patient's upper and/or lower limb, comprising:
a mobile support sized and shaped to receive a limb to be operated on;
extension means enabling application of a traction force on a proximal portion of the limb; and
means for measuring the traction force applied to the limb, the measuring means being linked to an automatic control device modifying, in the case of variation of the traction force applied to the limb by the extension means in relation to an extension index value stored in a memory, the traction force exerted on the limb until returning to the index value.

11. The traction system according to claim 10, wherein the support is mobile in rotation around an axis coinciding essentially with a pivoting axis of the limb.

12. The traction system according to claim 10, wherein the means for measuring the applied force is a dynamometer.

13. The traction system according to claim 12, wherein the dynamometer contains piezoelectric sensors.

14. The traction system according to claim 10, wherein the automatic control device comprises means for comparing the value of the force applied to the limb with a reference value of the force stored in the memory of the automatic control device.

15. The traction system according to claim 10, wherein the automatic control device comprises means for controlling displacement of the extension means in a manner such that the extension means exert on the limb the extension index value.

16. The traction system according to claim 10, further comprising visual and/or audio alarm means triggered upon detection by the automatic control system of a variation of the force applied to the limb in relation to a set index value.

17. An operating table comprising at least one traction system according to claim 10.

18. The operating table according to claim 17, wherein the limb is articulated around a pivot perpendicular to the table corresponding essentially to a position of the patient's hip.

19. A traction system applicable to a patient's upper and/or lower limb, comprising:
a mobile support sized and shaped to receive a limb to be operated on;
an extension device enabling application of a traction force on a proximal part of the limb; and
a measurement device connected to measure traction force applied to the limb, the measurement device being linked to an automatic control device modifying, in the case of variation of the traction force applied to the limb by the extension device in relation to an extension index value stored in a memory, the traction force exerted on the limb until returning to the index value.

20. The traction system according to claim 19, wherein the support is mobile in rotation around an axis coinciding essentially with a pivoting axis of the limb.

21. The traction system according to claim 19, wherein the measuring device is a dynamometer.

22. The traction system according to claim 21, wherein the dynamometer contains piezoelectric sensors.

23. The traction system according to claim 19, wherein the automatic control device comprises a comparing device that compares the value of the force applied to the limb with a reference value of the force stored in the memory of the automatic control device.

24. The traction system according to claim 19, wherein the automatic control device comprises a controller that controls displacement of the extension device in a manner such that the extension device exerts on the limb the extension index value.

25. The traction system according to claim 19, further comprising a visual and/or audio alarm that may be triggered upon detection by the automatic control system of a variation of the force applied to the limb in relation to a set index value.

26. An operating table comprising at least one traction system according to claim 19.

27. The operating table according to claim 26, wherein the limb is articulated around a pivot perpendicular to the table corresponding essentially to a position of the patient's hip.
